(19) United States
(12) Patent Application Publication
(10) Pub. No.: US 2023/0000770 A1
(43) Pub. Date: Jan. 5, 2023

(54) CANNABINOID NANOMICELLE PREPARATION AND METHOD FOR PREPARING SAME

(71) Applicant: HANYI BIOTECHNOLOGY (BEIJING) CO., LTD, Beijing (CN)

(72) Inventors: Xin TAN, Beijing (CN); Shubin WANG, Beijing (CN); Dekai FAN, Beijing (CN); Wuxing SUN, Beijing (CN); Junbo XING, Beijing (CN); Xuran ZHANG, Beijing (CN)

(73) Assignee: HANYI BIOTECHNOLOGY (BEIJING) CO., LTD, Beijing (CN)

(21) Appl. No.: 17/781,529

(22) PCT Filed: Nov. 12, 2020
(86) PCT No.: PCT/CN2020/128307

§ 371 Date: Jun. 1, 2022

(30) Foreign Application Priority Data
Dec. 6, 2019 (CN) 201911243459.3

(51) Int. Cl. A61K 9/107; A61K 31/047; A61K 47/10; A61K 47/34; A61K 47/26; A61K 47/12; A61K 47/18; A61K 9/19; A61K 9/00; A61K 9/20

(52) U.S. Cl. CPC A61K 9/1075; A61K 31/047; A61K 47/10; A61K 47/34; A61K 47/26; A61K 47/12; A61K 47/183; A61K 9/19; A61K 9/0075; A61K 9/2013; A61K 9/2018; A61K 9/2009; A61K 9/2031; A61K 9/2027; A61K 9/2054

(57) ABSTRACT

The invention discloses a cannabinoid nano-micelle preparation and a preparation method thereof. The cannabinoid nano-micelle preparation includes cannabinoid and an amphiphilic polymer, wherein the content of the cannabinoid is 1-40% by weight, the content of the amphiphilic polymer is 1-99%, and the preparation method includes the following steps: (1) preparing a cannabinoid nano-micelle solution from cannabinoid and an amphiphilic polymer; (2) drying the micellar solution obtained in the step (1) to obtain cannabinoid nano-micelle powder; and (3) preparing the cannabinoid nano-micelle powder obtained in the step (2) into the cannabinoid nano-micelle preparation. The cannabinoid nano-micelle preparation is high in effective component wrapping rate and transfer rate, high in drug loading capacity and high in stability, and a novel normal-temperature self-assembly technology is adopted, so that an active component cannabinoid is prevented from being degraded and discolored at high temperature; the bioavailability of the active ingredient is high, and a single dose can be reduced. Especially, a dry powder inhalant is high in in-vitro deposition rate and quick in inhalation effect, and can provide continuous and stable blood concentration.

### TECHNICAL FIELD

The invention relates to the technical field of pharmaceutical preparations, in particular to a cannabinoid nano-micelle preparation and a preparation method thereof, and particularly relates to a cannabinoid nano-micelle solid preparation and a preparation method thereof.

### BACKGROUND

Polymer micelles are core-shell type nanoparticles formed by self-assembly of a block copolymer in an aqueous solution. A hydrophobic drug can be embedded in a hydrophobic core, and a shell composed of hydrophilic chain segments forms a hydrated layer barrier to provide micelle stability. Its amphipathicity is realized by a hydrophilic block and a hydrophobic block. The hydrophilic block includes polyethylene glycol (PEG), polyethylene oxide (PEO) and polyvinylpyrrolidone (PVP); and the hydrophobic block includes polypropylene, polyamino acid, polylactic acid and the like. The polymer micelle is divided into an amphiphilic block copolymer, a triblock copolymer, a cross-linked copolymer and a graft copolymer. Polymer micelle solutions are mostly transparent solutions with a particle size of 10-100 nm, and a polymer micelle solution with a particle size of larger than 100 nm is opaque and is in an emulsion or suspension state. In the field of pharmaceutics, drug-loaded particles or drug nanocrystals with a size of 1-1000 nm are called nano-drugs.

Polymer nano-micelle drug loading has several unique advantages that: 1. the drug loading capacity is high, and the solubility of insoluble raw materials in water is greatly improved; 2. chemical structures of the raw materials can be solubilized without being changed; 3. slow release and controlled release of drugs can be realized, and stable blood concentration can be ensured in vivo for a long time; 4. the oral absorption of the drugs is promoted; 5. the targeting effect is achieved, and the small particle size is beneficial to penetrating into tumors with poor permeability; and 6. the lower CMC (critical micelle concentration) can be beneficial to keeping the micelle structure under the condition of being diluted by blood.

A nano-micelle preparation is generally a liquid oral preparation, particularly, almost no solid-state preparation exists in the fields of cannabinoid and monomer preparations, for example, the patent WO2019008178A1 describes a cannabinoid micelle liquid preparation.

Cannabinoid is a fat-soluble substance and is almost insoluble in water, so that the development of cannabinoid in the field of preparations is greatly restricted. Cannabis preparations are mostly oil solutions which are inconvenient to take, poor in stability and low in absorption bioavailability. An aqueous solution preparation of cannabinoid contains nano-emulsion, nano-capsules, nano-microspheres, cyclodextrin inclusion and the like, the drug loading capacity is generally low and is about 0-3%, the stability in an aqueous solution is poor, and the stability in gastric juice is poor.

### SUMMARY

In order to overcome the defects in the prior art, the invention provides a cannabinoid nano-micelle preparation and a preparation method thereof.

Specifically, the cannabinoid nano-micelle preparation includes cannabinoid, an amphiphilic polymer, and optionally, a pharmaceutically acceptable freeze-drying protective additive and a pH regulator, and is prepared by a method including the following steps:

(1) preparing a cannabinoid nano-micelle solution from cannabinoid and an amphiphilic polymer;

(2) diluting the micellar solution obtained in the step (1), and drying to obtain cannabinoid nano-micelle powder; and

(3) preparing the cannabinoid nano-micelle powder obtained in the step (2) into the cannabinoid nano-micelle preparation.

Specifically, in the above cannabinoid nano-micelle preparation, the content of the cannabinoid is 1-40% by weight (specifically, such as 1%, 5%, 10%, 15%, 20%, 25%, 30%, 35% and 40%).

Specifically, in the above cannabinoid nano-micelle preparation, the content of the amphiphilic polymer is 1-99% (specifically, such as 1%, 5%, 10%, 15%, 20%, 25%, 30%, 35%, 40%, 45%, 50%, 55%, 60%, 65%, 70%, 75%, 80%, 85%, 90%, 95% and 99%).

In one embodiment of the invention, the cannabinoid is selected from one or a combination of two or more of pure products of cannabidiol (CBD), cannabidivarin (CBDV), cannabigerol (CBG), cannabichromene (CBC), cannabinol (CBN), cannabidibutol (CBDB), cannabielsoin (CBE), cannabicyclol (CBL) and cannabinodiol (CBND), and the cannabinoid can be cannabinoid extracted from plants or synthetic cannabinoid, preferably the cannabinoid extracted from plants.

In another embodiment of the present invention, the cannabinoid is a cannabis extract, and includes one or a combination of two or more of CBD, CBDV, CBG, CBC, CBN, CBDB, CBE, CBL and CBND.

In one embodiment of the invention, the cannabinoid is CBD.

In one embodiment of the present invention, the amphiphilic polymer is vitamin E polyethylene glycol succinate (TPGS).

Specifically, the TPGS is selected from one or more of TPGS 200, TPGS 238, TPGS 400, TPGS 600, TPGS 1000, TPGS 2000, TPGS 3400, TPGS 3500, TPGS 4000 and TPGS 6000; and in one embodiment of the invention, the TPGS is the TPGS 1000.

In one embodiment of the present invention, the amphiphilic polymer is amphiphilic polyurethane.

Specifically, the amphiphilic polyurethane is an alternating copolymer which is obtained by alternately copolymerizing a hydrophilic chain segment, diisocyanate and the like, wherein the hydrophilic chain segment can be polyethylene glycol, polyoxyethylene and the like, and the diisocyanate is aliphatic or alicyclic diisocyanate, specifically, such as hexamethylene diisocyanate, trimethyl-1,6-hexamethylene diisocyanate, isophorone diisocyanate, cyclohexanedimethylene diisocyanate, 4,4-dicyclohexylmethane diisocyanate, 1,4-cyclohexane diisocyanate and the like.

Specifically, the amphiphilic polyurethane has a number-average molecular weight of 1000-50000 (specifically, such as 1000, 2000, 3000, 4000, 5000, 6000, 8000, 10000, 12000, 14000, 16000, 18000, 20000, 22000, 24000, 26000, 28000, 30000, 32000, 34000, 36000, 38000, 40000, 42000, 44000, 46000, 48000 and 50000).

In one embodiment of the invention, the hydrophilic chain segment in the amphiphilic polyurethane is polyethylene glycol, and the polyethylene glycol chain segment may have a number-average molecular weight of 200-12000 (specifically, such as 200, 400, 500, 600, 750, 1000, 2000, 4000, 5000, 6000, 8000, 10000 and 12000), in one embodiment of the invention, the polyethylene glycol chain segment may have a number-average molecular weight of 600-6000, and in one embodiment of the invention, the polyethylene glycol chain segment has a number-average molecular weight of 1000 or 1500.

In one embodiment of the present invention, the amphiphilic polymer is an amphiphilic polymer block copolymer, which is a block polymer composed of two or more polymers, and a hydrophilic part of the amphiphilic polymer can be polyethylene glycol, polyoxyethylene, povidone and the like, and a hydrophobic part of the amphiphilic polymer can be polyoxypropylene, polylactic acid, polystyrene, polycaprolactone, polyamino acid, poly(lactic-co-glycolic acid), polyacrylic acid and the like, such as one or a combination of two or more of poloxamer (PEO-PPO-PEO), a polylactic acid-polyethylene glycol-polylactic acid triblock copolymer (PLA-PEG-PLA), a polyethylene glycol-polyacrylic acid block copolymer (PEG-PAA), a polyethylene glycol-polyaspartic acid block copolymer (PEG-PASP), a polyethylene glycol-poly(lactic-co-glycolic acid) block copolymer (PEG-PLAG), a polyethylene glycol-polycaprolactone block copolymer (PEG-PCL), a polyethylene glycol-polylactic acid block copolymer (PEG-PLA/PTX), and a polyethylene glycol-polystyrene block copolymer (PEG-b-PS), and the like.

Specifically, the poloxamer is selected from one or a combination of two or more of poloxamer 188, poloxamer 338, poloxamer 407, poloxamer 124, poloxamer 215, poloxamer 237 and the like; and in one embodiment of the invention, the poloxamer is the poloxamer 188 or the poloxamer 338.

Specifically, in the polylactic acid-polyethylene glycol-polylactic acid triblock copolymer, a polyethylene glycol part may have a number-average molecular weight of 200-12000 (specifically, such as 200, 400, 500, 600, 750, 1000, 2000, 4000, 5000, 6000, 8000, 10000 and 12000), in one embodiment of the invention, the polyethylene glycol part may have a number-average molecular weight of 1000-6000, and in one embodiment of the invention, the polyethylene glycol part has a number-average molecular weight of 1000 or 2000; wherein a block ratio of PEG to LA is 1:5-50 (a molar ratio) (specifically, such as 1:5, 1:10, 1:12, 1:15, 1:20, 1:25, 1:30, 1:40 and 1:50); and in one embodiment of the invention, the block ratio of PEG to LA is 1:10 or 1:20.

Specifically, the polyethylene glycol-polyacrylic acid block copolymer, the polyethylene glycol-polyaspartic acid block copolymer, the polyethylene glycol-poly(lactic-co-glycolic acid) block copolymer, the polyethylene glycol-polycaprolactone block copolymer, the polyethylene glycol-polylactic acid block copolymer and the polyethylene glycol-polystyrene block copolymer have a number-average molecular weight of 500-50000 (specifically, such as 500, 1000, 2000, 3000, 4000, 5000, 6000, 8000, 10000, 12000, 14000, 16000, 18000, 20000, 22000, 24000, 26000, 28000, 30000, 32000, 34000, 36000, 38000, 40000, 42000, 44000, 46000, 48000 and 50000); wherein polyethylene glycol is polyethylene glycol monomethyl ether, the polyethylene glycol part may have a number-average molecular weight of 200-12000 (specifically, such as 200, 400, 500, 600, 750, 1000, 2000, 4000, 5000, 6000, 8000, 10000 and 12000), in one embodiment of the invention, the polyethylene glycol part may have a number-average molecular weight of 1000-6000, and in one embodiment of the invention, the polyethylene glycol part has a number-average molecular weight of 1000, 2000 or 5000.

In one embodiment of the invention, the cannabinoid nano-micelle preparation further includes a pharmaceutically acceptable freeze-drying protective agent.

Specifically, in the above cannabinoid nano-micelle preparation, the content of the freeze-drying protective agent is 1-10% (specifically, such as 1%, 2%, 3%, 4%, 5%, 6%, 7%, 8%, 9% and 10%).

Specifically, the freeze-drying protective agent is selected from one or more of lactose, mannitol, sorbitol, cyclodextrin, hydroxypropyl-β-cyclodextrin, EDTA-2Na, trehalose, glucose, xylitol and maltose;

In one embodiment of the invention, the cannabinoid nano-micelle preparation further includes a pH regulator.

Specifically, in the above cannabinoid nano-micelle preparation, the content of the pH regulator is 0.01-10% (specifically, such as 0.01%, 0.05%, 0.1%, 0.2%, 0.3%, 0.4%, 0.5%, 1%, 2%, 3%, 4%, 5%, 6%, 7%, 8%, 9% and 10%).

Specifically, the pH regulator is selected from one or more of citric acid, sodium citrate, tartaric acid, sodium tartrate, acetic acid, sodium acetate, sodium dihydrogen phosphate, disodium hydrogen phosphate, hydrochloric acid, lactic acid and sodium hydroxide.

In one embodiment of the invention, the step (1) includes the following steps:

(1-1) adding raw material components of the cannabinoid nano-micelle preparation into a solvent for swelling;

(1-2) shearing the mixture obtained in the step (1-1) by using a high-shear dispersing emulsifier to obtain an emulsion; and

(1-3) stirring the emulsion obtained in the step (1-2) to obtain a clear transparent nano-micelle solution.

In one embodiment of the invention, the solvent in the step (1-1) is water, such as water for injection.

Specifically, the usage amount of the solvent in the step (1-1) is 2-10 times (a mass ratio, specifically, such as 2, 3, 4, 5, 6, 7, 8, 9 and 10 times) of that of the raw material components.

Specifically, the swelling in the step (1-1) is performed for 0.1-2 h (specifically, such as 0.1 h, 0.5 h, 1 h, 1.5 h and 2 h).

Specifically, the shearing in the step (1-2) is performed for 1-30 min (specifically, such as 1 min, 5 min, 10 min, 15 min, 20 min, 25 min and 30 min).

Specifically, in the step (1-3), the stirring is performed at 15-35° C. (specifically, such as 15° C., 20° C., 25° C., 30° C. and 35° C.) for 1-48 h (specifically, such as 1 h, 6 h, 12 h, 24 h, 36 h and 48 h).

Specifically, the cannabinoid nano-micelle solution has a particle size of 1-500 nm (specifically, such as 1 nm, 10 nm, 50 nm, 100 nm, 150 nm, 200 nm, 250 nm, 300 nm, 350 nm, 400 nm, 450 nm and 500 nm).

Specifically, a solvent for diluting in the step (2) is water, especially water for injection.

Specifically, a dilution ratio in the step (2) is 2-100 (specifically, such as 2, 5, 10, 20, 30, 40, 50, 60, 70, 80, 90 and 100).

Specifically, the drying in the step (2) is atomization freeze drying or freeze drying.

Specifically, the atomization freeze-drying includes the steps of atomization, freezing and drying; wherein an atomization mode is selected from one or a combination of two or more of pneumatic atomization, pressure atomization, centrifugal atomization and ultrasonic atomization; the freezing is performed at −10° C. to −50° C. (specifically, such as −10° C., −15° C., −20° C., −25° C., −30° C., −35° C., −40° C., −45° C. and −50° C.); and the drying step is performed under a vacuum degree of 40 Pa or below (specifically, such as 40 Pa, 30 Pa, 20 Pa and 10 Pa) at 10-35° C. (specifically, such as 10° C., 15° C., 20° C., 25° C., 30° C. and 35° C.).

Specifically, the freeze drying includes the steps of pre-freezing, sublimation drying and desorption drying; wherein the pre-freezing is performed at −30° C. to −50° C. (specifically, such as −30° C., −35° C., −40° C., −45° C. and −50° C.) for 0.5-3 h (specifically, such as 0.5 h, 1 h, 2 h and 3 h) by controlling a vacuum degree to 1-100 Pa (1 Pa, 10 Pa, 20 Pa, 30 Pa, 40 Pa, 50 Pa, 60 Pa, 80 Pa and 100 Pa); the sublimation drying is performed at −20° C. to 10° C. (specifically, such as −20° C., −10° C., −5° C., 0° C., 5° C. and 10° C.) for 1-36 h (specifically, such as 1 h, 6 h, 12 h, 18 h, 24 h, 30 h and 36 h); and the desorption drying is performed at 10-30° C. (specifically, such as 10° C., 15° C., 20° C., 25° C. and 30° C.) for 1-24 h (specifically, such as 1 h, 6 h, 12 h, 18 h and 24 h).

Specifically, the cannabinoid nano-micelle preparation is a solid preparation or a semi-solid preparation.

In one embodiment of the invention, the cannabinoid nano-micelle preparation is an oral preparation, such as effervescent tablets, sustained release tablets, rapidly disintegrating tablets, dropping pills and the like.

In another embodiment of the invention, the cannabinoid nano-micelle preparation is a preparation for mucosal administration, such as a suppository and the like.

In another embodiment of the invention, the cannabinoid nano-micelle preparation is an inhalation preparation, such as a dry powder inhalant and the like.

In another embodiment of the invention, the cannabinoid nano-micelle preparation is a preparation for transdermal administration, such as a gel patch and the like.

In one embodiment of the invention, the cannabinoid nano-micelle preparation is an inhalant, and has a particle size of 1-5 μm (specifically, such as 1 μm, 2 μm, 3 μm, 4 μm and 5 μm), wherein the drying in the step (2) is atomization freeze drying.

In another embodiment of the invention, the cannabinoid nano-micelle preparation is effervescent tablets, sustained release tablets, rapidly disintegrating tablets, dropping pills, a suppository or a gel patch, wherein the drying in the step (2) is freeze drying.

The preparation method of the cannabinoid nano-micelle preparation provided by the invention includes the following steps:

(1) preparing a cannabinoid nano-micelle solution from cannabinoid and an amphiphilic polymer;

(2) diluting the micellar solution obtained in the step (1), and drying to obtain cannabinoid nano-micelle powder; and

(3) preparing the cannabinoid nano-micelle powder obtained in the step (2) into the cannabinoid nano-micelle preparation.

In one embodiment of the invention, the step (1) includes the following steps:

(1-1) adding raw material components of the cannabinoid nano-micelle preparation into a solvent for swelling;

(1-2) shearing the mixture obtained in the step (1-1) by using a high-shear dispersing emulsifier to obtain an emulsion; and

(1-3) stirring the emulsion obtained in the step (1-2) to obtain a clear transparent nano-micelle solution.

In one embodiment of the invention, the solvent in the step (1-1) is water, such as water for injection.

Specifically, the usage amount of the solvent in the step (1-1) is 2-10 times (a mass ratio, specifically, such as 2, 3, 4, 5, 6, 7, 8, 9 and 10 times) of that of the raw material components.

Specifically, the swelling in the step (1-1) is performed for 0.1-2 h (specifically, such as 0.1 h, 0.5 h, 1 h, 1.5 h and 2 h).

Specifically, the shearing in the step (1-2) is performed for 1-30 min (specifically, such as 1 min, 5 min, 10 min, 15 min, 20 min, 25 min and 30 min).

Specifically, in the step (1-3), the stirring is performed at 15-35° C. (specifically, such as 15° C., 20° C., 25° C., 30° C. and 35° C.) for 1-48 h (specifically, such as 1 h, 6 h, 12 h, 24 h, 36 h and 48 h).

Specifically, the cannabinoid nano-micelle solution has a particle size of 1-500 nm (specifically, such as 1 nm, 10 nm, 50 nm, 100 nm, 150 nm, 200 nm, 250 nm, 300 nm, 350 nm, 400 nm, 450 nm and 500 nm).

Specifically, a solvent for diluting in the step (2) is water, especially water for injection.

Specifically, a dilution ratio in the step (2) is 2-100 (specifically, such as 2, 5, 10, 20, 30, 40, 50, 60, 70, 80, 90 and 100).

Specifically, the drying in the step (2) is atomization freeze drying or freeze drying.

Specifically, the atomization freeze-drying includes the steps of atomization, freezing and drying; wherein an atomization mode is selected from one or a combination of two or more of pneumatic atomization, pressure atomization, centrifugal atomization and ultrasonic atomization; the freezing is performed at −10° C. to −50° C. (specifically, such as −10° C., −15° C., −20° C., −25° C., −30° C., −35° C., −40° C., −45° C. and −50° C.); and the drying step is performed under a vacuum degree of 40 Pa or below (specifically, such as 40 Pa, 30 Pa, 20 Pa and 10 Pa) at 10-35° C. (specifically, such as 10° C., 15° C., 20° C., 25° C., 30° C. and 35° C.).

Specifically, the freeze drying includes the steps of pre-freezing, sublimation drying and desorption drying; wherein the pre-freezing is performed at −30° C. to −50° C. (specifically, such as −30° C., −35° C., −40° C., −45° C. and −50° C.) for 0.5-3 h (specifically, such as 0.5 h, 1 h, 2 h and 3 h) by controlling a vacuum degree to 1-100 Pa (1 Pa, 10 Pa, 20 Pa, 30 Pa, 40 Pa, 50 Pa, 60 Pa, 80 Pa and 100 Pa); the sublimation drying is performed at −20° C. to 10° C. (specifically, such as −20° C., −10° C., −5° C., 0° C., 5° C. and 10° C.) for 1-36 h (specifically, such as 1 h, 6 h, 12 h, 18 h, 24 h, 30 h and 36 h); and the desorption drying is performed at 10-30° C. (specifically, such as 10° C., 15° C., 20° C., 25° C. and 30° C.) for 1-24 h (specifically, such as 1 h, 6 h, 12 h, 18 h and 24 h).

The invention further provides a method for preventing and/or treating diseases, including the step of administering an effective amount of the cannabinoid nano-micelle preparation provided by the invention to a subject in need thereof.

Specifically, in the method, the cannabinoid nano-micelle preparation has the above definition of the present invention.

Specifically, in the method, the diseases are indications of the corresponding cannabinoid in the cannabinoid nano-micelle preparation; such as pain (such as chronic pain), inflammation (such as dermatitis), tumors (such as glioma, leukemia, prostate cancer and the like), liver injury (such as ischemic liver injury and liver injury caused by chronic alcoholism), nervous system diseases (such as epilepsy, multiple sclerosis, Parkinson's disease, Alzheimer's disease and the like) and the like (see for example, “Rong Guo, Xuan Chen, Hongyan Guo, Review on Pharmacological Effects of Tetrahydrocannabinol and Cannabidiol, Research and Development of Natural Products, 2017, 29: 449-1453”).

In one embodiment of the invention, the subject is a mammal, in particular a human.

Specifically, the effective amount of the cannabinoid nano-micelle preparation depends on a plurality of factors including the age, weight, gender, natural health condition, and nutritional condition of patients, the taking time, a metabolic rate, severity of diseases, subjective judgment of diagnosis and treatment physicians and the like.

The invention further provides a health-care product, including the cannabinoid nano-micelle preparation provided by the invention.

The invention further provides a method for improving immunity and resisting oxidation, including the step of administering an effective amount of the health-care product to a subject in need thereof.

The cannabinoid nano-micelle preparation provided by the invention is high in effective component wrapping rate and transfer rate, high in drug loading capacity and high in stability, and a novel normal-temperature self-assembly technology is adopted, so that an active component cannabinoid is prevented from being degraded and discolored at high temperature; the bioavailability of the active ingredient is high, and a single dose can be reduced. Especially, the dry powder inhalant is high in drug loading capacity (10% or above), high in stability (the shelf life is 2-3 years), high in in-vitro deposition rate (an emptying rate can reach 95%, and an effective deposition rate can reach 45%), quick in inhalation effect and capable of providing continuous and stable blood concentration.

### DETAILED DESCRIPTION

Unless otherwise defined, all scientific and technical terms used in the invention have the same meaning as those generally understood by those skilled in the technical field to which the present invention relates, for example, the following abbreviations and corresponding substances appearing in the invention are as follows:

| | |
|---|---|
| CBDV | Cannabidivarin |
| CBD | Cannabidiol |
| CBG | Cannabigerol |
| CBN | Cannabinol |
| CBC | Cannabichromene |
| CBDB | 4-butyl-5′-metyl-2′-(prop-1-en-2-yl)-1′,2′,3′,4′- |
| | tetrahydro-(1,1′-bipheyl)-2,6-diol, Cannabidibutol |
| CBE | Cannabielsoin |
| CBL | Cannabicyclol |
| CBND | Cannabinodiol |
| TPGS | vitamin E polyethylene glycol succinate |
| Poloxamer | Poloxamer |
| PEG-PAA | Polyethylene glycol-polyacrylic acid block copolymer |
| PEG-PASP | Polyethylene glycol-polyaspartic acid block copolymer |
| PEG-PLAG | Polyethylene glycol-poly(lactic-co-glycolic acid) block |
| | copolymer |
| PEG-PCL | Polyethylene glycol-polycaprolactone block copolymer |
| PEG-PLA/PTX | Polyethylene glycol-polylactic acid block copolymer |
| PEG-b-PS | Polyethylene glycol-polystyrene block copolymer |
| SFD | Spray freeze drying |

In the invention, the term ‘cannabinoid’ refers to a class of secondary metabolites containing a molecular structure of alkyl and a monoterpene group unique to cannabis plants, for example, CBD, CBDV, CBG, CBC, CBN, CBDB, CBE, CBL, CBND and the like (as described in “Xuan Chen, Ming Yang, Hongyan Guo, Research Advances in Cannabinoids of Cannabis sativa, Botanical Newton, 2011, 46 (2): 197-205”).

In the invention, pure products of CBD, CBDV, CBG, CBC, CBN, CBDB, CBE, CBL and CBND refer to pure products of the compounds, especially corresponding commercially available products, and the purity of the compounds is at least 99.5% or above, especially 99.9% or above (the balance is impurities).

The technical solution of the invention is clearly and completely described in combination with the embodiments of the invention, and obviously, the described embodiments are only a part of the embodiments of the invention instead of all of the embodiments of the invention. Based on the embodiments of the present invention, other embodiments obtained by those of ordinary skill in the art without creative work all belong to the scope of protection of the present invention.

### Embodiment 1: Preparation of Cannabinoid Nano-micelle Solution

1. A specific formula of the cannabinoid nano-micelle solution is shown in the following table.

TABLE 1
Formula of cannabinoid nano-micelle solution

| | | TPGS | Poloxamer | Polyurethane | PEG 2000- | PEG 2000- | PEG 1000- |
|---|---|---|---|---|---|---|---|
| Formula | CBD | 1000 | 188 | 4000 | PAA 2000 | PASP 1000 | PLAG 3000 |
| 1 | 10% | 10% | 50% | 0 | 0 | 0 | 10% |
| 2 | 20% | 0 | 0 | 10% | 0 | 0 | 0 |
| 3 | 30% | 0 | 0 | 0 | 10% | 10% | 0 |
| 4 | 40% | 0 | 0 | 0 | 0 | 0 | 10% |
| 5 | 50% | 0 | 0 | 0 | 0 | 10% | 0 |
| 6 | 60% | 0 | 10% | 10% | 0 | 0 | 0 |

| | PEG 1000- | PEG 3400- | PEG 5000- | | | Citric | Tartaric |
|---|---|---|---|---|---|---|---|
| Formula | PCL 2000 | PLA/PTX 4000 | b-PS 5000 | Mannitol | Sorbitol | acid | acid |
| 1 | 0 | 0 | 0 | 5% | 5% | 1% | 0 |
| 2 | 50% | 0 | 0 | 5% | 5% | 0 | 1% |
| 3 | 0 | 30% | 0 | 5% | 5% | 1% | 0 |
| 4 | 10% | 0 | 20% | 5% | 5% | 0 | 1% |
| 5 | 0 | 10% | 10% | 5% | 5% | 1% | 0 |
| 6 | 0 | 0 | 0 | 5% | 5% | 0% | 1% |

| | | Hydroxypropyl- | EDTA-2 | | | |
|---|---|---|---|---|---|---|
| Formula | Cyclodextrin | β-cyclodextrin | Na | Trehalose | Leucine | Lactose |
| 1 | 0 | 5% | 0.1% | 1% | 0.5% | 2.4% |
| 2 | 5% | 0 | 0 | 1% | 0 | 3% |
| 3 | 0 | 5% | 0.1% | 1% | 0.5% | 2.4% |
| 4 | 5% | 0 | 0 | 1% | 0 | 3% |
| 5 | 0 | 5% | 0.1% | 1% | 0.5% | 2.4% |
| 6 | 5% | 0 | 0 | 1% | 0 | 3% |

2. Preparation process

(1) A first set of preparation processes (process 1)

The cannabinoid nano-micelle solution is prepared by feeding according to the proportion in formulae 1-6, specifically as follows:

a. swelling: 100g of raw materials are taken according to the formula in Table 1, and added into 800 g of water for swelling for 2 h;

b. emulsifying: the obtained mixture after swelling is sheared for 30 min by using a high-shear dispersing emulsifier at 50 Hz; and

c. stirring: the temperature of the emulsification system is controlled at 30° C., and stirring is performed for 48 h to obtain a clear transparent nano-micelle solution with a particle size of less than 500 nm.

The quality comparison of the cannabinoid nano-micelle solution obtained by the above preparation process is shown in the following table.

TABLE 2
Quality comparison of cannabinoid nano-micelle solution

| | Quality of corresponding solution | | | | |
|---|---|---|---|---|---|
| | | | | Effective | Effective |
| | | Solution | | component | ingredient |
| | Solution | particle | Solution | wrapping | transfer |
| Formula | clarity | size | color | rate % | rate % |
| 1 | Clear and | 0-500 nm | Colourless | 100% | 100% |
| | transparent | | | | |
| 2 | Clear and | 0-500 nm | Colourless | 100% | 100% |
| | transparent | | | | |
| 3 | Milky white | 0-500 nm | White | 90% | 100% |
| | liquid | | | | |
| 4 | Clear and | 0-500 nm | Colourless | 100% | 100% |
| | transparent | | | | |
| 5 | Clear and | 0-500 nm | Colourless | 100% | 100% |
| | transparent | | | | |
| 6 | Milky white | 0-500 nm | White | 90% | 100% |
| | liquid | | | | |

(2) A second set of preparation processes (process 2)

The cannabinoid nano-micelle solution is prepared by feeding according to the proportion in formulae 1-6, specifically as follows:

a. melting: TPGS 1000, Poloxamer 188, polyurethane 4000, PEG 2000-PAA 2000, PEG 2000-PASP 1000, PEG 1000-PLAG 3000, PEG 1000-PCL 2000, PEG 3400-PLA/PTX 4000 and PEG 5000-b-PS 5000 are added according to the formulae 1-6 in Table 1, and melted at 70° C., cannabinoid and a monomer thereof are added after melting, and uniform dispersing is performed to prepare a liquid 1;

b. dissolving: according to the formulas 1-6 in Table 1, mannitol, sorbitol, citric acid, tartaric acid, cyclodextrin, hydroxypropyl-β-cyclodextrin, EDTA-2Na, trehalose, leucine and lactose are dissolved with 8 times of water to prepare a liquid 2; and

c. mixing and dissolving: the liquid “1” and the liquid “2” are mixed and dissolved at 40° C., and slow stirring is performed for 12 h until a particle size of the solution is less than 500 nm.

The quality comparison of the cannabinoid nano-micelle solution obtained by the above preparation process is shown in the following table.

TABLE 3
Quality comparison of cannabinoid nano-micelle solution

| | Quality of corresponding solution | | | | |
|---|---|---|---|---|---|
| | | | | Effective | Effective |
| | | Solution | | component | ingredient |
| | Solution | particle | Solution | wrapping | transfer |
| Formula | clarity | size | color | rate % | rate % |
| 1 | Clear and | 0-500 nm | Colourless | 100% | 97% |
| | transparent | | | | |
| 2 | Clear and | 0-500 nm | Colourless | 100% | 97% |
| | transparent | | | | |
| 3 | Milky white | 0-500 nm | White | 90% | 96% |
| | liquid | | | | |
| 4 | Clear and | 0-500 nm | Colourless | 100% | 97% |
| | transparent | | | | |
| 5 | Clear and | 0-500 nm | Colourless | 100% | 97% |
| | transparent | | | | |
| 6 | Milky white | 0-500 nm | White | 90% | 96% |
| | liquid | | | | |

It can be seen from the results in Table 2 and Table 3 that under the same formula, the transfer rate of the cannabinoid nano-micelle prepared by the first set of processes is higher than that of the cannabinoid nano-micelle prepared by the second set of processes, which may be due to the melting step adopted in the second set of processes, which makes cannabinoid degrade.

### Embodiment 2: Preparation of Cannabinoid Nano-micelle Powder

A drying mode for preparing micelle powder includes vacuum drying, atomization freeze drying, nano spray drying, supercritical fluid technology and freeze drying, the formula 1 prepared by the first set of processes in Embodiment 1 is prepared into cannabinoid nano-micelle powder by the above drying techniques, respectively, and a quality comparison study is carried out, and a specific drying mode comparison is shown in Table 4.

Atomization freeze drying: the cannabinoid nano-micelle solution is sprayed into an atomization freeze dryer to produce the cannabinoid micelle micropowder, and a specific process is as follows:

a. atomization:

the nano-micelle solution prepared by the process 1 in Embodiment 1 is diluted to 100 times by using purified water, and subjected to atomization freeze drying by using an atomization freeze dryer with an atomization device adopting ultrasonic atomization.

b. freezing:

the solid content is 40% when pneumatic atomization, pressure atomization and centrifugal atomization are adopted, and the solid content is 10% when ultrasonic atomization is adopted;

Feeding flow rate: 10 ml/min;

Freezing temperature: −45° C.;

c. drying:

Vacuum degree: 40 Pa or below;

Drying temperature: 30° C.

A freeze-drying process: the cannabinoid nano-micelle solution is put into a freeze-drying oven for freezing at −40° C. for 3 h by controlling a vacuum degree to be less than 10 Pa, wherein primary drying is performed at −10° C. for 24 h; and secondary drying is performed at 25° C. for 12 h; and the dried material is sieved and subjected to straightening granulating through No.1-No.5 sieves.

The quality comparison of the powder prepared by the above drying processes is shown in the following table.

TABLE 4
Quality comparison of powder obtained by different drying processes

| | | | | | | | In-vitro | |
|---|---|---|---|---|---|---|---|---|
| | | | | | Particle size | | deposition determination | |
| Drying | Process | Powder | | Surface | distribution | | Emptying | Deposition |
| mode | controllability | hygroscopicity % | Solubility | morphology | d (0.9) μm | | rate % | rate % |
| Vacuum | Simple and | 2.0% | the solution | Needle-like | 50.1 | μm | 75% | 5% |
| drying | controllable | | is clear | | | | | |
| Atomization | Simple and | 1.0% | the solution | Spherical | 5 | μm | 95% | 45% |
| freeze drying | controllable | | is clear | | | | | |
| Nano spray | Simple and | 1.3% | the solution | Flaky | 20 | μm | 90% | 30% |
| drying | controllable | | is clear | | | | | |
| Supercritical | Complicated and | 2.0% | the solution | Flaky | 15 | μm | 89% | 29% |
| fluid | uncontrollable | | is clear | | | | | |
| Freeze drying | Simple and | 1.5% | the solution | Flaky | 48 | μm | 70% | 4% |
| | controllable | | is clear | | | | | |

A detection method of the above parameters is as follows:
1. Hygroscopicity: a certain amount of dry powder is weighed, and placed in a test tube (with an outer diameter of 50 mm and a height of 15 mm), the test tube which is opened is placed for 24 h in an environment with a temperature of 25° C. and a relative humidity of 70%, the test tube is weighed and a moisture absorption weight gain is calculated.
2. Solubility: 0.2 g of dry drug powder is taken, and put into a test tube, 5 mL of pure water is added, oscillating is performed, and the dissolution condition of the dry drug powder is observed. If the dry drug powder can be dissolved to obtain a clear solution, the drug has good solubility and can be used as a pulmonary inhalation preparation, and if the dry drug powder cannot be dissolved, the drug has poor water solubility and is not suitable for being used as a pulmonary inhalation preparation.
3. Surface morphology: the contact points among spherical particles are minimum, the dispersibility is best, and more plane contact points exist among particles in the shapes of flakes, needles and the like, so that the flowability is relatively poor.
4. Particle size distribution: represented by d (0.9) μm, and the particle size of 90% of particles is less than this particle size. The particles of 1-5 μm can enter the lung through the respiratory airflow, and a good lung deposition rate is achieved.
5. In-vitro deposition rate determination: the in-vitro deposition rate determination is carried out by using NGI at 60 L/min, including an emptying rate % and an effective deposition rate %, the emptying rate of a solid powder inhalation preparation is not lower than 90%, and the deposition rate is greater than 30%.

Result analysis: through the comparative analysis of several drying modes from the aspects of process controllability, powder hygroscopicity, solubility, surface morphology, particle size distribution, in-vitro deposition rate determination and other indicators, it is determined that atomization freeze drying has more advantages in inhalation preparation than other powders, and freeze-dried powder is more suitable for the preparation of other solid preparations.

### Embodiment 3: Cannabinoid Nano-micelle Inhalation Preparation

The powder obtained by atomization freeze drying in Embodiment 2 has a particle size of 1-5 μm, the surface morphology is spherical, the fluidity is better, the emptying rate is 95%, the deposition rate is 45%, the powder is suitable for being used as inhalation powder, and the powder is filled into No.2-No.00 capsules, vesicles and reservoirs or can be directly used for administration by inhalation.

The bioavailability comparison between the cannabinoid nano-micelle inhalation powder and the micellar solution prepared according to the method described in the embodiments of the patent application WO2019008178A1 is shown in the following table.

TABLE 5
Bioavailability comparison

| | Bioavailability |
|---|---|
| Item | AUC |
| Inhalation powder prepared by this solution | 60% |
| Micellar solution prepared according to the patent | 18% |
| WO2019008178A1 | |

### Embodiment 4: Preparation of Cannabinoid Nano-micelle Effervescent Tablets

1. Formula: cannabinoid micelle powder (powder obtained by a freeze drying process in Embodiment 2), DL-tartaric acid, citric acid, sodium bicarbonate, mannitol, lactose, aspartame, xylitol and polyethylene glycol. A specific formula is shown in the following table.

TABLE 6
Formula of cannabinoid nano-micelle effervescent tablets

| | Cannabinoid | DL- | | | | | | | |
|---|---|---|---|---|---|---|---|---|---|
| | micelle | tartaric | Citric | Sodium | | | | | Polyethylene |
| Formula | powder | acid % | acid % | bicarbonate % | Mannitol % | Lactose % | Aspartame % | Xylitol % | glycol % |
| 1 | 10% | 20% | 5% | 25% | 5% | 20% | 1% | 5% | 9% |
| 2 | 20% | 20% | 5% | 25% | 0 | 15% | 1% | 5% | 9% |
| 3 | 30% | 20% | 5% | 25% | 0 | 10% | 1% | 4% | 5% |

Specification: the tablet weight is 2 g.

2. Preparation process

According to the formula in Table 6, the cannabinoid micelle powder, DL-tartaric acid, citric acid, mannitol and polyethylene glycol are prepared into particles 1 by using a fluidized bed or by wet granulation; sodium bicarbonate, aspartame, lactose, xylitol and polyethylene glycol are prepared into particles 2 by using a fluidized bed or by wet granulation; and the particles 1, the particles 2 and the cannabinoid micelle powder are uniformly mixed, and pressed into effervescent tablets, wherein the tablet weight is controlled to be 2 g.

The quality comparison of the effervescent tablets of different formulas is shown in the following table.

TABLE 7
Quality comparison of cannabinoid
nano-micelle effervescent tablets

| | | | Solution state | |
|---|---|---|---|---|
| | Disintegration | Dispersion | after effer- | Content |
| Formula | time/min | uniformity | vescence | stability |
| 1 | 2 | Qualified | Clear and | The content is |
| | | | transparent | stable after |
| | | | | tablets are |
| | | | | subjected to |
| | | | | accelerated test |
| | | | | for 6 months |
| 2 | 3 | Qualified | Clear and | The content is |
| | | | transparent | stable after |
| | | | | tablets are |
| | | | | subjected to |
| | | | | accelerated test |
| | | | | for 6 months |
| 3 | 5 | Qualified | Clear and | The content is |
| | | | transparent | stable after |
| | | | | tablets are |
| | | | | subjected to |
| | | | | accelerated test |
| | | | | for 6 months |

Result analysis: the effervescent tablets all meet the requirements of the effervescent tablet quality standard, the effervescent tablets are quickly dissolved in water and convenient to use and carry, the dissolving speed of cannabinoid powder can be increased, and the effervescent solution is a cannabinoid nano-micelle solution which can be directly taken.

### Embodiment 5: Preparation of Cannabinoid Nano-micelle Sustained Release Tablets

1. Formula: cannabinoid micelle powder (powder obtained by a freeze drying process in Embodiment 2), hydroxypropyl methylcellulose, polyvinylpyrrolidone, ethyl cellulose, lactose, superfine silica powder and magnesium stearate. A specific formula is shown in the following table.

TABLE 8
Formula of cannabinoid nano-micelle sustained release tablets

| | Cannabinoid | | | | | Superfine | |
|---|---|---|---|---|---|---|---|
| | micelle | Hydroxypropyl | Polyvinyl- | Ethyl | | silica | Magnesium |
| Formula | powder | methylcellulose | pyrrolidone | cellulose | Lactose | powder | stearate |
| 1 | 10% | 50% | 9% | 9% | 20% | 1% | 1% |
| 2 | 30% | 20% | 10% | 10% | 28% | 1% | 1% |
| 3 | 50% | 20% | 10% | 6% | 12% | 1% | 1% |

2. Preparation process: according to the formula in Table 8, hydroxypropyl methylcellulose, polyvinylpyrrolidone, ethyl cellulose, lactose and tartaric acid are added, and granulated with a certain amount of absolute ethyl alcohol, cannabinoid micelle powder, superfine silica powder and magnesium stearate are added, uniform mixing is performed, and the obtained mixture is pressed into 1 g of sustained release tablets.

The quality comparison of the sustained-release tablets of different formulas is shown in the following table.

TABLE 9
Quality comparison of cannabinoid nano-
micelle sustained release tablets

| | | Tablet | | | |
|---|---|---|---|---|---|
| | | weight | Hardness | In vitro | |
| Formula | Properties | variation | kg/cm² | dissolution | Stability |
| 1 | White to | ±2.5% | 9.0 | Linear | Stable after |
| | brown | | | release | being |
| | tablets | | | with a | subjected to |
| | | | | cumulative | accelerated |
| | | | | release rate | test for 6 |
| | | | | of 92% | months |
| 2 | White to | ±2.0% | 9.2 | Linear | Stable after |
| | brown | | | release | being |
| | tablets | | | with a | subjected to |
| | | | | cumulative | accelerated |
| | | | | release | test for 6 |
| | | | | degree | months |
| | | | | of 90% | |
| 3 | White to | ±2.6% | 9.1 | Linear | Stable after |
| | brown | | | release | being |
| | tablets | | | with a | subjected to |
| | | | | cumulative | accelerated |
| | | | | release rate | test for 6 |
| | | | | of 91% | months |

In vitro dissolution: 6 cannabinoid nano-micelle sustained release tablets in a same batch are taken and put into a rotating basket, a rotating speed of the rotating basket is set to be 120 rpm, the temperature is set to be 37.0 ± 0.5° C., and the cumulative release rate of linear release of a sample within 12 h is 90% or more by taking 1000 mL of fresh degassed distilled water as a dissolution medium.

The properties, tablet weight variation, hardness, in vitro dissolution and stability of the sustained-release tablets in the formulae all meet the requirements, cannabinoid can be slowly released in the intestinal tract, cannabinoid nano-micelles can slowly release cannabinoid after entering the blood, the dual sustained-release effect is achieved, the cannabinoid can maintain stable blood concentration in vivo for a long time, and the sustained-release tablets take effect on chronic pains for a long time.

### Embodiment 6: Preparation of Cannabinoid Nano-micelle Orally Disintegrating Tablets

1. Formula: cannabinoid micelle powder (powder obtained by a freeze drying process in Embodiment 2), L-HPC, lactose, microcrystalline cellulose, pregelatinized starch, superfine silica powder and magnesium stearate. A specific formula is shown in the following table.

TABLE 10
Formula of cannabinoid nano-micelle orally disintegrating tablets

| | Cannabinoid | | | | | Superfine | |
|---|---|---|---|---|---|---|---|
| | micelle | | | Microcrystalline | Pregelatinized | silica | Magnesium |
| Formula | powder | L-HPC | Lactose | cellulose | starch | powder | stearate |
| 1 | 10% | 30% | 30% | 19% | 10% | 0.5% | 0.5% |
| 2 | 30% | 30% | 20% | 10% | 9% | 0.5% | 0.5% |
| 3 | 50% | 25% | 15% | 5% | 4% | 0.5% | 0.5% |

2. Preparation process

The materials are prepared into granules through a wet method or one-step granulation, and the granules are pressed into 0.5 g of rapidly disintegrating tablets.

The quality comparison of the quick-release tablets of different formulas is shown in the following table.

TABLE 11
Mass comparison of cannabinoid nano-micelle quick-release tablets

| | | | Tablet | |
|---|---|---|---|---|
| | | Disintegration | weight | |
| Formula | Properties | time S | variation | Stability |
| 1 | White to | 40S | ±2.5% | Stable after |
| | brown | | | being subjected |
| | tablets | | | to accelerated |
| | | | | test for 6 months |
| 2 | White to | 45S | ±2.3% | Stable after |
| | brown | | | being subjected |
| | tablets | | | to accelerated |
| | | | | test for 6 months |
| 3 | White to | 48S | ±2.2% | Stable after |
| | brown | | | being subjected |
| | tablets | | | to accelerated |
| | | | | test for 6 months |

The properties, tablet weight variation, disintegration time and stability of the orally disintegrating tablets of the three formulas all meet requirements, the orally disintegrating tablets can be rapidly disintegrated in the mouth and absorbed into blood under the tongue after being taken in the mouth, the first-pass effect is avoided, and the orally disintegrating tablets are convenient for patients with dysphagia to take.

### Embodiment 7: Preparation of Cannabinoid Nano-micelle Gel

1. Formula: cannabinoid micelle powder (powder obtained by a freeze drying process in Embodiment 2), carbomer, glycerol, propylene glycol, triethanolamine, EDTA-2Na and ethylparaben. A specific formula is shown in the following table.

TABLE 12
Formula of cannabinoid nano-micelle gel

| | Cannabinoid | | | | | | |
|---|---|---|---|---|---|---|---|
| | micelle | | | Propylene | | | |
| Formula | powder | Carbomer | Glycerol | glycol | Triethanolamine | EDTA-2Na | Ethylparaben |
| 1 | 10% | 10% | 47% | 28.9% | 3% | 0.1% | 1% |
| 2 | 30% | 12% | 30% | 23.9% | 3% | 0.1% | 1% |
| 3 | 50% | 13% | 20% | 11.9% | 4% | 0.1% | 1% |

2. Preparation process: according to the formula in Table 12, 100 times of water is added into carbomer for swelling, triethanolamine is added to form a gel matrix, then the cannabinoid micelle powder, glycerol, propylene glycol, EDTA-2Na and ethylparaben are added, and uniform stirring is performed to obtain the cannabinoid nano-micelle gel.

The mass comparison of gel of different formulas is shown in the following table.

TABLE 13
Quality comparison of cannabinoid nano-micelle gel

| Formula | Properties | Uniformity | Stability |
|---|---|---|---|
| 1 | Colorless to | Evenly | Stable after being |
| | brown paste | dispersed and | subjected to |
| | solid | delicate | accelerated test |
| | | | for 6 months |
| 2 | Colorless to | Evenly | Stable after being |
| | brown paste | dispersed and | subjected to |
| | solid | delicate | accelerated test |
| | | | for 6 months |
| 3 | Colorless to | Evenly | Stable after being |
| | brown paste | dispersed and | subjected to |
| | solid | delicate | accelerated test |
| | | | for 6 months |

The properties, uniformity and stability of the gel in the three formulas meet the requirements, and the gel can be directly smeared on the affected part to achieve the effects of treating dermatitis and joint chronic pain, removing acnes and freckles and the like.

### Embodiment 8: Preparation of Cannabinoid Nano-micelle Dropping Pills

1. Formula: cannabinoid micelle powder (powder obtained by a freeze drying process in Embodiment 2), polyethylene glycol 4000 and polyethylene glycol 6000. A specific formula is shown in the following table.

TABLE 14
Formula of cannabinoid nano-micelle dropping pills

| | Cannabinoid | Polyethylene | Polyethylene | |
|---|---|---|---|---|
| | micelle | glycol | glycol | Water |
| Formula | powder | 4000% | 6000% | % |
| 1 | 10% | 40% | 45% | 5% |
| 2 | 30% | 25% | 30% | 5% |
| 3 | 50% | 20% | 25% | 5% |

2. Preparation process: according to the formula in Table 14, the cannabinoid micelle powder, polyethylene glycol 4000, polyethylene glycol 6000 and purified water are heated at 70° C. to be melted to prepare slurry 1, the condensation temperature of paraffin oil is controlled to be 10° C., the slurry 1 is put into a pill dropping machine, dropping is performed to obtain dropping pills of 50 mg/pill, and the paraffin oil on the surfaces of the dropping pills is removed by using oil absorption cotton to obtain the cannabinoid nano-micelle dropping pills.

The quality comparison of the dropping pills of different formulas is shown in the following table.

TABLE 15
Quality comparison of cannabinoid nano-micelle dropping pills

| | | Pill weight | Disintegration | | |
|---|---|---|---|---|---|
| Formula | Properties | difference | time/min | | Stability |
| 1 | White to | ±8% | 10 | min | Stable after being |
| | yellowish- | | | | subjected to |
| | brown | | | | accelerated test |
| | pills | | | | for 6 months |
| 2 | White to | ±7% | 9 | min | Stable after being |
| | yellowish- | | | | subjected to |
| | brown | | | | accelerated test |
| | pills | | | | for 6 months |
| 3 | White to | ±8% | 11 | min | Stable after being |
| | yellowish- | | | | subjected to |
| | brown | | | | accelerated test |
| | pills | | | | for 6 months |

The properties, pill weight difference, disintegration time and stability of the dropping pills in the three formulas all meet the requirements, and the dropping pills can be taken orally or sublingually, and is quick in effect and lasting in blood concentration.

### Embodiment 9: Preparation of Cannabinoid Nano-micelle Suppository

1. Formula: cannabinoid micelle powder (powder obtained by a freeze drying process in Embodiment 2), cocoa butter, mixed fatty acid glyceride, polyethylene glycol, and ethylparaben. A specific formula is shown in the following table.

TABLE 16
Formula of cannabinoid nano-micelle suppository

| | Cannabinoid | | Mixed fatty | Poly- | |
|---|---|---|---|---|---|
| | micelle | Cocoa | acid | ethylene | Ethyl- |
| Formula | powder | butter | glyceride | glycol | paraben |
| 1 | 10% | 50% | 30% | 9.5% | 0.5% |
| 2 | 30% | 40% | 20% | 9.5% | 0.5% |
| 3 | 50% | 25% | 20% | 4.5% | 0.5% |

2. Preparation process: according to the formula in the above table, the cannabinoid micelle powder, cocoa butter, mixed fatty acid glyceride, polyethylene glycol and ethylparaben are melted at 40-60° C., and the melted material is put into a suppository preparation machine to prepare suppositories such as spherical suppositories, bullet suppositories, capsule suppositories and the like.

The quality comparison of the suppositories of different formulas is shown in the following table.

TABLE 17
Quality comparison of cannabinoid nano-micelle suppositories

| | | Weight | Melting | |
|---|---|---|---|---|
| Formula | Properties | difference % | time/min | Stability |
| 1 | White to | 4.1% | 25 min | Stable after being |
| | brown | | | subjected to |
| | suppository | | | accelerated test |
| | | | | for 6 months |
| 2 | White to | 4.0% | 26 min | Stable after being |
| | brown | | | subjected to |
| | suppository | | | accelerated test |
| | | | | for 6 months |
| 3 | White to | 4.5% | 27 min | Stable after being |
| | brown | | | subjected to |
| | suppository | | | accelerated test |
| | | | | for 6 months |

The properties, the weight difference, the melting time and the stability of the suppositories in the three formulas all meet the requirements, and the suppositories can directly reach focuses to achieve the anti-inflammatory effect or be used for systemic administration to treat chronic pains, so that the application range of cannabinoid is expanded.

The above is only better embodiments of the invention, and is not intended to limit the invention, and any modification, equivalent replacement and the like made within the spirit and principle of the invention should be included in the protection scope of the invention.

1. A cannabinoid nano-micelle preparation, comprising cannabinoid and an amphiphilic polymer; wherein the content of the cannabinoid is 1-40% by weight, and the content of the amphiphilic polymer is 1-99%, and a preparation method of the preparation comprises the following steps:
(1) preparing a cannabinoid nano-micelle solution from cannabinoid and an amphiphilic polymer;
(2) diluting the micellar solution obtained in the step (1), and drying to obtain cannabinoid nano-micelle powder; and
(3) preparing the cannabinoid nano-micelle powder obtained in the step (2) into the cannabinoid nano-micelle preparation.

2. The cannabinoid nano-micelle preparation according to claim 1, characterized in that the cannabinoid is selected from one or a combination of two or more of pure products of cannabidiol (CBD), cannabidivarin (CBDV), cannabigerol (CBG), cannabichromene (CBC), cannabinol (CBN), cannabidibutol (CBDB), cannabielsoin (CBE), cannabicyclol (CBL) and cannabinodiol (CBND); or,
the cannabinoid is a cannabis extract, and comprises one or a combination of two or more of CBD, CBDV, CBG, CBC, CBN, CBDB, CBE, CBL and CBND.

3. The cannabinoid nano-micelle preparation according to claim 1, characterized in that the amphiphilic polymer is vitamin E polyethylene glycol succinate.

4. The cannabinoid nano-micelle preparation according to claim 1, characterized in that the amphiphilic polymer is amphiphilic polyurethane;
the amphiphilic polyurethane is obtained by alternately copolymerizing a polyethylene glycol chain segment and a raw material comprising diisocyanate.

5. The cannabinoid nano-micelle preparation according to claim 1, characterized in that the amphiphilic polymer is selected from one or a combination of two or more of poloxamer, a polylactic acid-polyethylene glycol-polylactic acid triblock copolymer, a polyethylene glycol-polyacrylic acid block copolymer, a polyethylene glycol-polyaspartic acid block copolymer, a polyethylene glycol-poly(lactic-co-glycolic acid) block copolymer, a polyethylene glycol-polycaprolactone block copolymer, a polyethylene glycol-polylactic acid block copolymer and a polyethylene glycol-polystyrene block copolymer.

6. The cannabinoid nano-micelle preparation according to claim 1, characterized in that the cannabinoid nano-micelle preparation further comprises a pharmaceutically acceptable freeze-drying protective agent, wherein the content of the freeze-drying protective agent is 1-10%, and the freeze-drying protective agent is selected from one or more of lactose, mannitol, sorbitol, cyclodextrin, hydroxypropyl-β-cyclodextrin, EDTA-2Na, trehalose, glucose, xylitol and maltose.

7. The cannabinoid nano-micelle preparation according to claim 6, characterized in that the cannabinoid nano-micelle preparation further comprises a pH regulator, wherein the content of the pH regulator is 0.01-10%, and the pH regulator is selected from one or more of citric acid, sodium citrate, tartaric acid, sodium tartrate, acetic acid, sodium acetate, sodium dihydrogen phosphate, disodium hydrogen phosphate, hydrochloric acid, lactic acid and sodium hydroxide.

8. The cannabinoid nano-micelle preparation according to claim 1, characterized in that the step (1) comprises the following steps:
(1-1) adding raw material components of the cannabinoid nano-micelle preparation into a solvent for swelling;
(1-2) shearing the mixture obtained in the step (1-1) by using a high-shear dispersing emulsifier to obtain an emulsion; and
(1-3) stirring the emulsion obtained in the step (1-2) to obtain a clear transparent nano-micelle solution.

9. The cannabinoid nano-micelle preparation according to claim 8, characterized in that a mass ratio of the solvent to the raw material components in the step (1-1) is (2-10):1;
the swelling in the step (1-1) is performed for 0.1-2 h;
the shearing in the step (1-2) is performed for 1-30 min; and
the stirring in the step (1-3) is performed at 15-35° C.

10. The cannabinoid nano-micelle preparation according to claim 8, characterized in that the cannabinoid nano-micelle solution has a particle size of 1-500 nm.

11. The cannabinoid nano-micelle preparation according to claim 1, characterized in that a dilution ratio in the step (2) is 2-100.

12. The cannabinoid nano-micelle preparation according to claim 1, characterized in that the drying in the step (2) is atomization freeze drying or freeze drying.

13. The cannabinoid nano-micelle preparation according to claim 1, characterized in that the preparation is a solid preparation or a semi-solid preparation.

14. The cannabinoid nano-micelle preparation according to claim 1, characterized in that the preparation is an oral preparation, a preparation for mucosal administration or a preparation for transdermal administration.

15. A preparation method of the cannabinoid nano-micelle preparation according to claim 1, comprising the following steps:
(1) preparing a cannabinoid nano-micelle solution from cannabinoid and an amphiphilic polymer;
(2) diluting the micellar solution obtained in the step (1), and drying to obtain cannabinoid nano-micelle powder; and
(3) preparing the cannabinoid nano-micelle powder obtained in the step (2) into the cannabinoid nano-micelle preparation;
wherein the step (1) comprises the following steps:
(1-1) adding raw material components of the cannabinoid nano-micelle preparation according to any one of claims 1-9 into a solvent for swelling;
(1-2) shearing the mixture obtained in the step (1-1) by using a high-shear dispersing emulsifier to obtain an emulsion; and
(1-3) stirring the emulsion obtained in the step (1-2) to obtain a clear transparent nano-micelle solution.

16. The preparation method according to claim 15, characterized in that a mass ratio of the solvent to the raw material components in the step (1-1) is (2-10):1;
the swelling in the step (1-1) is performed for 0.1-2 h;
the shearing in the step (1-2) is performed for 1-30 min; and
the stirring in the step (1-3) is performed at 15-35° C.

17. The preparation method according to claim 15, characterized in that a dilution ratio in the step (2) is 2-100.

18. The preparation method according to claim 15, characterized in that the drying in the step (2) is atomization freeze drying or freeze drying.

19. The preparation method according to claim 18, characterized in that the atomization freeze drying comprises the steps of atomization, freezing and drying,
wherein an atomization mode is selected from one or a combination of two or more of pneumatic atomization, pressure atomization, centrifugal atomization and ultrasonic atomization;
the freezing is performed at −10° C. to −50° C.; and
the drying step is performed under a vacuum degree of 40 Pa or below at 10-35° C.

20. The preparation method according to claim 18, characterized in that the freeze drying comprises the steps of pre-freezing, sublimation drying and desorption drying;
wherein the pre-freezing step is performed at −30° C. to −50° C. for 0.5-3 h by controlling a vacuum degree to 1-100 Pa;
the sublimation drying is performed at −20° C. to 10° C. for 1-36 h; and
the desorption drying is performed at 10-30° C. for 1-24 h.
